(19) United States
(12) Patent Application Publication
(10) Pub. No.: US 2014/0128378 A1
(43) Pub. Date: May 8, 2014

(54) METHODS OF TREATING EPILEPTOGENESIS

(71) Applicant: UCB Pharma GmbH, Monheim (DE)

(72) Inventor: Thomas STÖHR, Monheim (DE)

(73) Assignee: UCB Pharma GmbH, Monheim (DE)

(21) Appl. No.: 14/154,410

(22) Filed: Jan. 14, 2014

Related U.S. Application Data
(63) Continuation of application No. 12/304,012, filed on Jun. 7, 2010.

(30) Foreign Application Priority Data
Jun. 30, 2006 (EP) 06013655.3

(51) Int. Cl. A61K 31/165; A61K 31/5513

(52) U.S. Cl. CPC A61K 31/165; A61K 31/5513
USPC 514/220; 514/616; 514/221

(57) ABSTRACT

The present invention is directed to the use of a class of peptide Compounds for prevention, alleviation or/and treatment of refractory Status epilepticus.

The present application claims the priority of the U.S. provisional application U.S. 60/813,967 of 15 Jun. 2006, which is included herein by reference. The present application also claims the priority of EP 06 013 655.3 of 30 Jun. 2006, which is included herein by reference.

The present invention is directed to the use of a class of peptide compounds for treating refractory status epilepticus (SE) or a related condition.

In particular, the present invention is directed to the use of a combination of a class of peptide compounds and a drug used in the treatment of SE, such as benzodiazepines, anticonvulsants, or barbiturates, in particular a benzodiazepine for treating refractory status epilepticus or/and a condition related to refractory status epilepticus, such as epileptogenesis or epileptogenesis caused by refractory status epilepticus.

U.S. Pat. No. 5,378,729 describes peptide compounds exhibiting central nervous system (CNS) activity and are useful in the treatment of epilepsy, nervous anxiety, psychosis and insomnia. EP 1 541 138 describes peptide compounds useful for treatment of status epilepticus. However, neither of these patents describes the use of these compounds for the treatment of refractory status epilepticus or/and a related condition, such as epileptogenesis or epileptogenesis caused by refractory status epilepticus.

Seizures are the consequence of a paroxysmal brain dysfunction related to excessive neuronal activity that leads to an alteration of behaviour or consciousness. Epilepsy represents the recurrence of two or more unprovoked seizures and represents a chronic brain disease. About 0.5% of the population suffers epilepsy, and up to 10% of the population will suffer at least one seizure during their life-time.

There are two major types of seizures: partial or focal seizures, which originate in a location in the brain, but can spread in the course of the event; and generalized seizures, which can affect both hemispheres simultaneously.

Partial seizures are manifested in multiple ways (confusion, automatic body movements, hallucinations, etc.) depending on the area of the brain that is affected, and if they spread in the brain can end up in a generalized tonic-clonic event (a convulsion). A complex partial seizure is a type of partial seizure originating in the temporal lobe and characterized by impairment of consciousness, often preceded by a hallucinatory aura. If a partial seizure spreads in the brain it can end up as a generalized seizure, for example a tonic-clonic convulsion There are several types of generalized seizures: convulsive (tonic-clonic, tonic, clonic, myoclonic) and non-convulsive (absences, atonic). Typically all kinds of seizures last up to a few minutes, generally less than five minutes. Convulsive seizures, particularly tonic-clonic events, typically result in impairment of consciousness.

Status epilepticus is currently defined as a seizure that lasts for 30 or more minutes, or a series of consecutive seizures that occur for 30 or more minutes during which the subject does not completely recover consciousness. Many clinicians and many recent major research articles, however, consider a patient to be in status if seizures last more than 5 minutes.

For purposes of the present application, SE will be understood to mean any epileptic event in which a generalized or partial seizure lasts longer than 5 minutes, or in which a series of generalized or partial seizures occur during a period longer than 5 minutes without full recovery of consciousness between seizures.

There are two main types of status epilepticus: generalized (convulsive and non-convulsive) and focal. The generalized convulsive status is the most severe type and is associated with high morbidity and mortality. Status epilepticus can occur in patients with prior epilepsy diagnosis. However, the onset of status is more frequent in subjects without previous epilepsy and is often related to a severe and acute brain disease (for example, an encephalitis or a stroke). In addition to these, a variety of conditions including hypoglycemia, hyperthermia, drug overdose and alcohol or drug withdrawal can be a cause of SE. Thus, anticonvulsant activity of a compound or combination, for example in models for or patients with complex partial seizures, is not necessarily predictive for activity against SE. SE is not only a life threatening disease but also causes neuronal cell loss and epileptogenesis.

Status epilepticus or related conditions represent an emergency and pharmacological treatment should preferably be carried out using intravenous medication. Drugs currently used for initial treatment include intravenous benzodiazepines (for example diazepam or lorazepam), anticonvulsants (for example phenyloin, fosphenyloin or valproic acid) and barbiturates (for example phenobarbital). Intravenous valproic acid has also been used. Rectal or intramuscular administration routes may also be used. Despite these first line treatments, over 40% of the subjects will not respond. Under these circumstances, pharmacological coma, induced for example by pentobarbital, thiopental, propofol, high dose of midazolam or other benzodiazepines is needed to treat status.

Recent population-based studies indicate that status epilepticus still carries an acute mortality of 27% in adults, and there is a general consensus that standard drugs used are unsatisfactory. While they work relatively well if given very early in the course of status epilepticus, they lose their efficacy quickly if seizures continue for more than half an hour. Barbiturates and other GABAergic drugs never become totally inactive, but can require such high doses that toxic side effects prevent a fully effective treatment. In animal models for instance, the potency of benzodiazepines can decrease 20 times within 30 min of self-sustaining status epilepticus. Other anticonvulsants, such as phenyloin, also lose potency, but more slowly.

Thus, early initiation of anticonvulsants is crucial in current treatment of status epilepticus and to prevent its long-term consequences, e.g. neuronal cell loss and epileptogenesis (for review, see Chen J W, Wasterlain C G, Lancet Neurol 2006, 5:246-56).

Optimal treatment of refractory SE and the prevention of its consequences as defined herein have not been established.

The prevalence of epilepsy following status epilepticus is three time higher than following a single ‘normal’ seizure Indicating the status epilepticus is highly epileptogenic (for review see Chen and Wasterlain Lancet Neurology 2006). So far no drug has shown inhibition of epileptogenesis induced by status epilepticus in humans.

Most frequently used is e.g. administration of midazolam (see Claassen et al., Neurology 57 (2001), 1036-1042), propofol, or pentobarbital (Stecker et al., Epilepsia 39 (1998), 18-26). A meta-analysis based on a literature review by Claassen et al. (Epilepsia 43 (2002), 146-153) of the response of patients in refractory SE to treatment with midazolam, propofol or pentobarbital revealed low treatment failure but high incidence of hypotension for pentobarbital, a high number of breakthrough seizures for midazolam and similar high numbers of withdrawal seizures for all therapies. Thus, there is an unmet need for further therapy options of refractory SE.

While seizures are the common symptom for both epilepsy and status epilepticus, status epilepticus frequently occurs in subjects not suffering from epilepsy. A variety of other diseases such as stroke, brain trauma or encephalitis or a variety of conditions like hypoglycaemia, hyperthermia, drug overdose or alcohol or drug withdrawal can be the cause of status epilepticus. Thus, the anticonvulsant activity in models for or patients with complex partial seizures is not necessarily predictive for an activity against status epilepticus.

Current anti-epileptic drugs are believed to work through diverse mechanisms of action, including for example altering neuronal impulse propagation via interaction with voltage-gated sodium, calcium or potassium channels, or affecting neural transmission either by potentiating inhibitory GABA (gamma-aminobutyric acid) systems or by inhibition of excitatory glutamate systems.

(R)-2-acetamido-N-benzyl-3-methoxypropionamide (lacosamide, previously called SPM 927 or harkoseride) is a functionalized amino acid initially synthesized as an anticonvulsant. Lacosamide appears to be more potent and effective as compared to other clinically effective anticonvulsant drugs (phenyloin, carbamazepine) when it was evaluated in several anticonvulsant animal models.

Lacosamide is a representative of a class of compounds embraced by Formulae (I), (II), or/and (III) herein, which are generally well tolerated. Thus, especially where the refractory nature of SE cannot be overcome by increasing the dose of a standard anti-epileptic drug because of the risk of unacceptable adverse side effects, the present method can be advantageous.

The use of compounds of Formulae (I), (II), or/and (III) for treatment of refractory status epilepticus has not been reported. Thus, the present invention concerns the use of the compounds of Formula (I), (II), or/and (III) for the preparation of a pharmaceutical composition for the prevention, alleviation or/and treatment of refractory epileptic condition, particularly refractory status epilepticus, or/and a condition related to refractory status epilepticus.

Furthermore there is provided a method for treating refractory status epilepticus or/and a related condition in a subject, comprising administering to the subject at least one compound of Formula (I), (II), or/and (III).

Long-term consequences of status epilepticus including refractory status epilepticus are neuronal damage e.g. cell loss in the hippocampus and epileptogenesis i.e. the occurrence of spontaneous seizures at several months to years following the first status epilepticus event.

“Condition related to status epilepticus” or “condition related to refractory status epilepticus” as used herein includes a condition caused by status epilepticus, for example epileptogenesis or neuronal cell loss. “Epileptogenesis” as used herein includes the development of epilepsy, such as chronic epilepsy, or an epileptic condition as described herein.

Surprisingly, it was found that lacosamide (50 mg/kg) administered 40 min after onset of self sustaining status epilepticus (SSSE) in perforant path stimulated rats effected a 40% reduction in seizure frequency and cumulative seizure duration. The percentage of rats developing chronic epilepsy following 6 months was reduced from 100% to 30%. Similarly the number of seizures per week after six months was reduced by 60%. 6 months after induction of SSSE 100% of vehicle treated animals developed spontaneous recurrent seizures with an average of 110 seizures per week. Following treatment with lacosamide (30-50 mg/kg) only 30% of rats developed spontaneous recurrent seizures and seizure frequency reduced by 60%. Literature data indicate that the self sustaining status epilepticus in this model is responsive to treatment with benzodiazepines or hydantoins (phenyloin and fosphenyloin) within the first 10 min of status epilepticus but later becomes refractory to those agents (Mazarati et al. 1999, Neurosci. Lett. 265:187-190).

In the rat lithium/pilocarpine model of SE, lacosamide treatment (50 mg/kg) 10 minutes after onset of status epilepticus still resulted in reduced motor seizure symptoms while standard anti-status drugs were completely ineffective. Although standard anti-status drugs e.g. benzodiazepines were ineffective, a combination of 50 mg/kg lacosamide administered 10 min after onset of status epilepticus and 20 mg/kg diazepam administered 15 min after onset of status epilepticus was surprisingly superior over lacosamide alone, since full seizures control was achieved in all rats by this combination treatment.

From these experimental findings, it is concluded that the compounds of the present invention, in particular lacosamide, or a combination of the compounds of the present invention, in particular of lacosamide, with a further drug used in the treatment of SE, such as benzodiazepines, anticonvulsants or barbiturates, in particular a benzodiazepine such as diazepam, lorazepam, midazolam, clonazepam, clorazepate or/and clobazan are suitable for the treatment of a long-lasting status epilepticus which has become refractory or which is or becomes refractory in the course of its duration.

The compounds of the present invention of Formulae (I), (II) or/and (III), in particular lacosamide, are well tolerated, which is an advantage over other commonly used therapeutics for treatment of refractory epileptic conditions such as refractory status epilepticus.

Without being bound by theory, the mode of action of the compounds of Formulae (I), (II) or/and (III) differs from that of common antiepileptic drugs. Ion channels are not affected by the compounds of the present invention in a manner comparable to other known antiepileptic drugs, whereas GABA-induced currents are potentiated, but no direct interaction with any known GABA receptor subtype is observed. Glutamate induced currents are attenuated but the compounds do not directly interact with any known glutamate receptor subtype.

As used herein, “epileptic condition” refers to a disease state including status epilepticus, an epileptic seizure, a repetitive seizure or/and a seizure cluster.

As used herein, “refractory epileptic condition” refers to a disease state including status epilepticus, an epileptic seizure, a repetitive seizure or/and a seizure cluster which is at least partially resistant or substantially resistant against one or more drugs employed in the treatment of status epilepticus or/and epilepsy. In particular, these drugs are different from the compounds of Formulae I, II, or/and III as defined herein. More particular, it refers to a disease state which is at least partially refractory or substantially refractory against at least one drug selected from benzodiazepines, barbiturates, and anticonvulsants different from the compounds of Formulae I, II or/and III as defined herein, particularly selected from diazepam, lorazepam, midazolam, phenobarbital, carbamazepine, phenyloin, fosphenyloin, oxcarbazepine, lamotrigine, gabapentin, pregabalin, valproic acid, pentobarbital, thiopental, propofol and pharmaceutically acceptable salts thereof.

A refractory status epilepticus or a related condition in a particular patient may be present a priori, or may be caused by the duration of status epilepticus.

In certain embodiments of the present invention, a refractory epileptic condition comprises status epilepticus, an epileptic seizure, a repetitive seizure or/and a seizure cluster, which has become at least partially refractory due to its duration for at least about 10 min, at least about 15 min, at least about 20 min, at least about 30 min, at least about 45 min, or at least about 60 min, preferably at least about 30 min, at least about 45 min, or at least about 60 min.

In certain embodiments, SE treated by a method of the present invention is initially responsive to treatment with one or more drugs employed in the treatment of status epilepticus or/and epilepsy as described herein, but becomes at least partially refractory when it lasts for at least about 10 minutes, for example at least about 15 minutes, at least about 20 minutes, at least about 30 minutes, at least about 45 minutes or at least about 60 minutes.

The compounds of the present invention, in particular lacosamide, may be used in a first line treatment of a refractory condition considered to be refractory due to the duration of the disease state as defined above. More particularly, the pharmaceutical composition of the present invention is suitable for a first line treatment of refractory status epilepticus or a related condition.

The compounds of the present invention may also be used in a second line treatment of a refractory condition, wherein therapy resistance has already become apparent in a preceding treatment, in particular in a treatment with benzodiazepines, barbiturates, and anticonvulsants different from the compounds of the present invention, in particular phenyloin, phosphenyloin, and valproate. More particularly, the pharmaceutical composition of the present invention is suitable for a second line treatment of refractory status epilepticus or a related condition.

The seizures in refractory status epilepticus may be focal seizures or/and may be generalized seizures. The generalized seizures may be convulsive generalized seizures, such as tonic-clonic, tonic, clonic, or myoclonic seizures, or may be non-convulsive seizures, such as absences or atonic seizures. Typically, the refractory status epilepticus involves at least partial loss of conciousness.

Thus, in one embodiment, the refractory status epilepticus or a related condition comprises focal seizures or/and generalized seizures. In another embodiment, the refractory status epilepticus or a related condition comprises convulsive seizures, such as tonic-clonic, tonic, clonic, or myoclonic seizures, or/and non-convulsive seizures, such as absences or atonic seizures.

In yet another embodiment, the refractory status epilepticus or a related condition comprises acute repetitive seizures or/and seizure clusters.

Yet another aspect of the present invention is a pharmaceutical composition comprising at least one compound of Formulae I, II or/and III as defined herein, preferably lacosamide, for the prevention, alleviation or/and treatment of refractory epileptic condition such as refractory status epilepticus or a related condition.

As discussed above, a combination of lacosamide and diazepam administered 15 min after onset of status epilepticus was surprisingly found to be superior to lacosamide alone in an animal model of status epilepticus, since full seizures control was achieved in all rats by this combination treatment. Diazepam alone administered at this point in time was found to be ineffective. Thus, the compounds of Formulae I, II or/and III may also be administered together with a further active agent, e.g. an antiepileptic drug, particularly a benzodiazepine drug.

A further aspect of the present invention is a pharmaceutical composition comprising
(a) at least one compound of Formulae I, II or/and III as defined herein, preferably lacosamide, and
(b) at least one further active agent, e.g. a benzodiazepine, preferably diazepam, lorazepam, midazolam, clonazepam, clorazepate or/and clobazan.

In a particular embodiment, the further active agent is an anti-epileptic agent, for example comprising at least one benzodiazepine, barbiturate, and/or anticonvulsive other than a compound of Formula (I).

In one embodiment a pharmaceutical composition comprises one of the specific combinations lacosamide and diazepam, lacosamide and lorazepam, or lacosamide and midazolam. In this embodiment, the compound of (a) is lacosamide and the compound of (b) is diazepam, lorazepam or midazolam.

As used herein, “benzodiazepine” includes any benzodiazepine employed for treatment of status epilepticus, including diazepam, lorazepam, midazolam, clonazepam, clorazepate and clobazan. Preferred benzodiazepines are diazepam, lorazepam, or/and midazolam. Further antiepileptic drugs also include anticonvulsants or/and barbiturates.

The pharmaceutical composition comprising the combination of agents (a) and (b) as defined above is beneficial for the prevention, alleviation or/and treatment of any epileptic condition, particularly a refractory condition as defined above.

Refractory epileptic conditions treatable by the method of the present embodiment include not only refractory SE as defined above, but also epileptic seizures, repetitive seizures and seizure clusters that are at least partially resistant or substantially resistant to treatment with anti-epileptic drugs such as benzodiazepines, barbiturates and/or anticonvulsants other than compounds of Formula (I), including seizures that do not necessarily involve loss of consciousness.

Yet another aspect of the present invention is the use of at least one compound of Formulae I, II, or/and III as defined herein, in particular lacosamide, for the preparation of a pharmaceutical composition for the prevention, alleviation or/and treatment of epileptogenesis. In this use, “epileptogenesis” includes all embodiments of epileptogenesis as described herein.

Yet another aspect of the present invention is a method for the prevention, alleviation or/and treatment of epileptogenesis comprising administering to a subject in need thereof at least one compound of Formulae I, II, or/and III as defined herein, in particular lacosamide. In this method, “epileptogenesis” includes all embodiments of epileptogenesis as described herein. In one embodiment, epileptogenesis is related to status epilepticus, such as refractory status epilepticus.

In one embodiment, the method for the prevention, alleviation or/and treatment of epileptogenesis of the present invention comprises administering a further active agent, particularly a benzodiazepine such as diazepam, lorazepam, or/and midazolam.

In another embodiment, in the method for the prevention, alleviation or/and treatment of epileptogenesis of the present invention, the at least one compound of Formulae I, II, or/and III as defined herein, in particular lacosamide, is administered after onset of status epilepticus. In the method for the prevention, alleviation or/and treatment of epileptogenesis of the present invention, the at least one compound of Formulae I, II, or/and III may be administered at least about 10 minutes or at least 30 min after onset of status epilepticus. The compound of Formulae I, II, or/and III in particular lacosamide, may be administered at a dose of about 50 to about 500 mg. The compound of Formulae I, II, or/and III, in particular lacosamide, may be administered intravenously.

The compound of Formulae I, II or/and III and the further active agent, e.g. the benzodiazepine may be formulated in one pharmaceutical preparation (single dosage form) for administration at the same time or may be formulated in two or more distinct preparations (separate dosage forms) for simultaneous or/and subsequent administration. The two distinct preparations in the separate dosage forms may be administered by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, compound of Formulae I, II or/and III and the benzodiazepine. In another example, the compound of Formulae I, II or/and III and the further active agent, e.g. the benzodiazepine are separately packaged and available for sale independently of one another, but are co-marketed or co-promoted for use according to the invention. The separate dose forms may also be presented to a subject separately and independently, for use according to the invention.

In a further embodiment, the pharmaceutical composition comprises a single dosage form comprising at least one compound of Formulae I, II or/and III and at least one further active agent, e.g. a benzodiazepine.

There is still further provided a pharmaceutical composition comprising at least one compound of Formula I, II or/and III at least one benzodiazepine, and at least one pharmaceutically acceptable excipient.

In another embodiment, the pharmaceutical composition of the present invention comprises separate dosage forms comprising
(i) a first composition comprising at least one compound of Formulae I, II or/and III, and
(ii) a second composition comprising at least one further active agent, e.g. a benzodiazepine.

In yet another embodiment of the present invention, the second composition (ii) comprising the at least one further active agent may be a commercially available composition.

The pharmaceutical composition of the present invention is in one embodiment prepared for administration in mammals, preferably in humans.

The pharmaceutical composition of the present invention, in particular the composition comprising at least one compound of the present invention and at least one benzodiazepine may be prepared for administration at least about 10 min, at least about 15 min, at least about 20 min, at least about 40 min, at least about 45 min, or at least about 60 min, preferably at least about 30 min, at least about 45 min, or at least about 60 min after the onset of a status epilepticus or a related condition.

The pharmaceutical composition of the present invention, in particular the composition comprising at least one compound of the present invention and at least one benzodiazepine may be prepared for administration to patients suffering from refractory status epilepticus, which is at least partially or substantially resistant against drugs employed in the treatment of status epilepticus, particularly selected from diazepam, lorazepam, midazolam, phenobarbital, carbamazepine, phenyloin, fosphenyloin, oxcarbazepine, lamotrigine, gabapentin, pregabalin, valproic acid, pentobarbital, thiopental, propofol and pharmaceutically acceptable salts thereof.

The pharmaceutical composition of the present invention comprising (a) at least one compound of Formulae (I), (II) or/and (III) and (b) at least one further active agent, e.g. a benzodiazepine may be prepared for the prevention, alleviation or/and treatment of a status epilepticus (including refractory and non-refractory status epilepticus) or/and epilepsy.

Yet another aspect of the present invention is a method for the prevention, alleviation or/and treatment of a refractory epileptic condition, wherein the method comprises administering to a subject in need thereof at least one compound of Formulae I, II or/and III, in particular lacosamide, optionally together with a further active agent, e.g. a benzodiazepine.

In one embodiment, the method further comprises administering a second active agent, in particular an anti-epileptic agent selected from benzodiazepines, barbiturates and anticonvulsive agents other than a compound of Formula (I).

A further aspect of the present invention is a method for the prevention, alleviation or/and treatment of an epileptic condition, wherein the method comprises co-administering to a subject in need thereof at least one compound of Formulae I, II or/and III, in particular lacosamide, and a benzodiazepine, in particular diazepam, lorazepam, or/and midazolam, in therapeutically effective amounts. One embodiment comprises the co-administration of one of the specific combinations lacosamide and diazepam, lacosamide and lorazepam, or lacosamide and midazolam.

According to any of the above embodiments, an illustrative compound of Formula (I) is lacosamide, (R)-2-acetamido-N-benzyl-3-methoxypropionamide.

In the method of the present invention, the at least one compound of the present invention, alone or in combination with at least one further compound, e.g. a benzodiazepine, is preferably administered to a subject in need thereof after the onset of the condition, e.g. about 10 min, about 15 min, about 20 min, about 30 min, about 40 min, about 45 min, about 60 min or more after the onset of the condition.

The term “co-administration” refers to a plurality of agents that, when administered to a subject together or separately, are co-active in bringing therapeutic benefit to the subject. Such co-administration is also referred to as “combination”, “combination therapy,” “co-therapy,” “adjunctive therapy” or “add-on therapy.” For example, one agent can potentiate or enhance the therapeutic effect of another, or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

Co-administration comprises administration of the combination of the agents in amounts sufficient to achieve or/and maintain therapeutically effective concentrations, e.g. plasma concentrations, in the subject in need thereof. Co-administration comprises simultaneous or/and subsequent administration. Simultaneous administration comprises administration of the agents as a single or as different compositions.

Sequential administration normally comprises administration of the compound of Formulas (I), (II) or (III), for example lacosamide, and the second active agent within an interval of up to about 90 minutes, for example up to about 60, up to about 45, up to about 40, up to about 30, up to about 20, up to about 10 or up to about 5 minutes. The administration interval can depend on the dosage forms and routes of administration of the agents. The compound of Formulas (I), (II) or (III), for example lacosamide, may be administered first, or the second active agent may be administered first.

When the method further comprises administration of a second active agent, as in the present embodiment, the compound of Formulas (I), (II) or (III), for example lacosamide, and the second active agent, e.g., a benzodiazepine, may be formulated in one pharmaceutical preparation (single dosage form) for administration at the same time, or alternatively may be formulated in two or more distinct preparations (separate dosage forms) for simultaneous and/or sequential administration. Separate dosage forms may be administered by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, a compound of Formulas (I), (II) or (III) and a benzodiazepine. In another example, a compound of Formulas (I), (II) or (III) and a benzodiazepine are separately packaged and available for sale independently of one another, but are co-marketed or co-promoted for use according to the invention. Separate dosage forms may also be presented to a subject separately and independently, for use according to the invention.

In yet another embodiment of the present invention, a therapeutic combination comprises at least one compound of Formulas (I), (II) or (III), for example lacosamide, and at least one benzodiazepine. The combination can be used for treatment of any medical condition responsive thereto, including without limitation epileptic conditions such as SE, for example where such conditions are or become refractory as described above.

Any benzodiazepine can be used in the combination, particularly a benzodiazepine having anti-epileptic activity such as one or more of diazepam, lorazepam, midazolam, clonazepam, clorazepate and clobazan.

The compound of Formulas (I), (II) or (III), for example lacosamide, and the benzodiazepine, for example diazepam, lorazepam or midazolam, are present in the combination in therapeutically effective total and relative amounts. For example, in a combination comprising lacosamide and diazepam, lacosamide can be present in an amount providing a dose of about 50 to about 500 mg and diazepam in an amount providing a dose of about 10 to about 100 mg.

Each of the components of the therapeutic combination can be provided in a pharmaceutical composition adapted for the desired route of delivery, for example as an injectable composition where the components are to be administered intravenously. Each pharmaceutical composition comprises one or more excipient ingredients as more fully described above. The benzodiazepine, for example, can be provided in the form of a commercially available pharmaceutical composition.

Alternatively, the compound of Formulas (I), (II) or (III), for example lacosamide, and the benzodiazepine can be provided in a single pharmaceutical composition adapted for a particular route of administration.

Accordingly, in yet another embodiment of the present invention, a pharmaceutical composition comprises
(a) at least one compound of Formulas (I), (II) or (III), for example lacosamide;
(b) at least one benzodiazepine, for example diazepam, lorazepam, midazolam, clonazepam, clorazepate and/or clobazan; and
(c) at least one pharmaceutically acceptable excipient.

As used herein, “pharmaceutically acceptable excipient” includes any and all such materials mentioned above, and any and all solvents, dispersion media, coatings, antibacterial and antifungal agents, isotonic and absorption delaying agents for pharmaceutically active substances known in the art. Except insofar as any conventional excipient is incompatible with one or both active ingredients, its use in a pharmaceutical composition of the present embodiment is contemplated. One or more further active agents, in addition to those specified above, can optionally be present.

Since status epilepticus is an emergency situation, immediate administration of the compound of Formulae I, II or/and III and optionally a further active agent, e.g. a benzodiazepine is required. Subsequent administration comprises administration of the compound of Formulae I, II or/and III and the further active agent within an interval of up to 5 min, up to 10 min, up to 20 min, up to 30 min, up to 40 min, up to 60 min, or up to 90 min. The administration interval of the compound of Formulae I, II or/and III and the further active agent may depend on the dosage forms. The compound of Formulae I, II or/and III may be administered first, or the further active agent may be administered first.

The compound according to the invention has the general Formula (I)

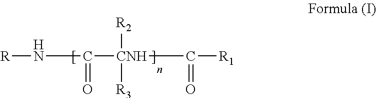

wherein
R is hydrogen, alkyl, alkenyl, alkynyl, aryl, aryl alkyl, heterocyclic, heterocyclic alkyl, alkyl heterocyclic, cycloalkyl or cycloalkyl alkyl, and R is unsubstituted or is substituted with at least one electron withdrawing group, or/and at least one electron donating group;
R₁ is hydrogen or alkyl, alkenyl, alkynyl, aryl alkyl, aryl, heterocyclic alkyl, alkyl heterocyclic, heterocyclic, cycloalkyl, cycloalkyl alkyl, each unsubstituted or substituted with at least one electron donating group or/and at least one electron withdrawing group;
and
R₂ and R₃ are independently hydrogen, alkyl, alkenyl, alkynyl, alkoxy, alkoxyalkyl, aryl alkyl, aryl, halo, heterocyclic, heterocyclic alkyl, alkyl heterocyclic, cycloalkyl, cycloalkyl alkyl, or Z—Y wherein R₂ and R₃ may be unsubstituted or substituted with at least one electron withdrawing group or/and at least one electron donating group;
Z is O, S, S(O)ₐ, NR₄, NR′₆, PR₄ or a chemical bond;
Y is hydrogen, alkyl, aryl, aryl alkyl, alkenyl, alkynyl, halo, heterocyclic, heterocyclic alkyl, alkyl heterocyclic and Y may be unsubstituted or substituted with at least one electron donating group or/and at least one electron withdrawing group, provided that when Y is halo, Z is a chemical bond, or

ZY taken together is NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR₄NR₅R₇ or N+R₅R₆R₇,

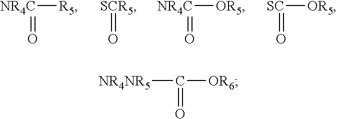

R′₆ is hydrogen, alkyl, alkenyl, or alkynyl and which may be unsubstituted or substituted with at least one electron withdrawing group or/and at least one electron donating group;
R₄, R₅ and R₆ are independently hydrogen, alkyl, aryl, aryl alkyl, alkenyl, or alkynyl, wherein R₄, R₅ and R₆ may independently be unsubstituted or substituted with at least one electron withdrawing group or/and at least one electron donating group;
R₇ is R₆ or COOR₈ or COR₈, which R₇ may be unsubstituted or substituted with at least one electron withdrawing group or/and at least one electron donating group;
R₈ is hydrogen or alkyl, or aryl alkyl, and the aryl or alkyl group may be unsubstituted or substituted with at least one electron withdrawing group or/and at least one electron donating group; and
n is 1-4; and
a is 1-3
or a pharmaceutically acceptable salt thereof.

In one embodiment, the compound of Formula (I) has the general Formula (II),

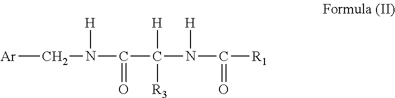

wherein
Ar is aryl which is unsubstituted or substituted with at least one electron donating group or/and at least one electron withdrawing group, preferably halo, more preferably fluoro;
R₁ is alkyl, preferably alkyl containing 1-3 carbon atoms, more preferably methyl; and
R₃ is as defined herein
   or a pharmaceutically acceptable salt thereof.

In another embodiment, the compound of Formulae (I) or/and (II) has the general Formula (III),

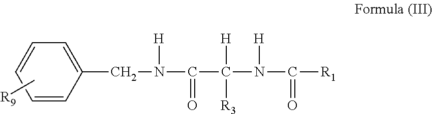

wherein
R₉ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, carbalkoxy, amino, alkylamino, dialkylamino, aryloxy, mercapto, alkylthio, alkylmercapto, and disulfide;
R₃ is selected from the group consisting of hydrogen, alkyl, arylalkyl, alkoxy, alkoxyalkyl, aryl, heterocyclic, heterocyclic alkyl, N-alkoxy-N-alkylamino, N-alkoxyamino, and N-carbalkoxy; and
R₁ is alkyl, preferably alkyl containing 1 to 3 carbon atoms, more preferably methyl
   or a pharmaceutically acceptable salt thereof.

The compounds utilized in the present invention may contain one or more asymmetric carbons and may exist in racemic and optically active forms. The configuration around each asymmetric carbon can be either the D or L form. It is well known in the art that the configuration around a chiral carbon atoms can also be described as R or S in the Cahn-Prelog-lngold nomenclature system. All of the various configurations around each asymmetric carbon, including the various enantiomers and diastereomers as well as racemic mixtures and mixtures of enantiomers, diastereomers or both are contemplated by the present invention.

As used herein, the term configuration particularly refers to the configuration around the carbon atom to which R₂ and R₃ or H and R₃ are attached, even though other chiral centers may be present in the molecule. Therefore, when referring to a particular configuration, such as D or L, it is to be understood to mean the D or L stereoisomer at the carbon atom to which R₂ and R₃ or H and R₃ are attached. However, it also includes all possible enantiomers and diastereomers at other chiral centers, if any, present in the compound.

The compounds of the present invention are directed to all the optical isomers, i.e., the compounds of the present invention are either the L-stereoisomer or the D-stereoisomer (at the carbon atom to which R₂ and R₃ or H and R₃ are attached). These stereoisomers may be found in mixtures of the L and D stereoisomer, e.g., racemic mixtures. The D stereoisomer is preferred.

It is preferred that the compounds of Formula (I) are in the R configuration. It is also preferred that the compounds of Formula (II) are in the R configuration. It is also preferred that the compounds of Formula (III) are in the R configuration.

It is preferred that the compounds of Formulae (I), (II) or/and (III) in the R configuration are substantially enantiopure. As used herein, the term “substantially enantiopure” refers to a content of the R enantiomer of at least 99.5%. This corresponds to an enantiomeric excess (ee) of 99%. The respective quantities of R and S enantiomer may be determined by chiral column chromatography, e.g. by HPLC with “ChiralPak” as chiral, stationary phase.

In one embodiment the compound, for example lacosamide, is substantially enantiopure.

As used herein, the term “substantially enantiopure” means having at least 88%, preferably at 90%, more preferably at least 95, 96, 97, 98, or 99% enantiomeric purity.

The term “alkyl” (alone or in combination with another term(s)) means a straight- or branched-chain saturated hydrocarbyl substituent preferably containing from 1 to about 20 carbon atoms (C₁-C₂₀-alkyl), more preferably from 1 to about 8 carbon atoms (C₁-C₈-alkyl), even more preferably from 1 to about 6 carbon atoms (C₁-C₆-alkyl), and most preferably from 1 to 3 carbon atoms (C₁-C₃-alkyl). The alkyl groups include methyl, ethyl, propyl, isopropyl, butyl, isobutyl, tertiary butyl, amyl, hexyl, and the like. Further, alkyl groups also include halogenated alkyl groups up to perhalogenation, e.g. trifluoromethyl, if not indicated otherwise.

The term “alkoxy” (alone or in combination with another term(s)) refers to —O-alkyl and means a straight- or branched-chain alkoxy substituent preferably containing from 1 to about 20 carbon atoms (C₁-C₂₀-alkoxy), more preferably from 1 to about 8 carbon atoms (C₁-C₈-alkoxy), even more preferably from 1 to about 6 carbon atoms (C₁-C₆-alkoxy), and most preferably from 1 to 3 carbon atoms (C₁-C₃-alkoxy). The alkoxy groups include methoxy, ethoxy, propoxy, butoxy, isobutoxy, tert-butoxy, pentoxy, hexoxy and the like. Further, alkoxy groups include halogenated alkoxy groups up to perhalogenation, if not indicated otherwise.

The term “alkoxyalkyl” refers to an alkyl group substituted with at least one alkoxy group. The alkoxyalkyl groups include methoxymethyl (—CH₂—OCH₃) groups, methoxyethyl (—CH₂—CH₂—OCH₃) groups, ethoxymethyl (—CH₂—O—CH₂CH₃) groups and the like.

The term “N-alkoxyamino” refers to amino groups substituted with one or two alkoxy groups, e.g. —NH—N(OCH₃)₂.

The term “N-alkoxy-N-alkylamino” refers to amino groups substituted with an alkoxy group and an alkyl group, e.g. —N(CH₃)(OCH₃), —N(CH₃)(OCH₂—CH₃) and the like.

The term “N-carbalkoxy” refers to amino groups substituted with a carbalkoxy group, e.g. —NH(C(O)—O—CH₃), —NH(C(O)O—CH₂—CH₃).

The term “aryl”, when used alone or in combination with other term(s), refers to an aromatic group which contains from 6 up to 18 ring carbon atoms (C₆-C₁₈-aryl), preferably from 6 up to 10 ring carbon atoms (C₆-C₁₈-aryl), and includes polynuclear aromatics. The aryl groups may be monocyclic, bicyclic, tricyclic or polycyclic and may be fused rings. A polynuclear aromatic compound as used herein, is meant to encompass bicyclic and tricyclic fused aromatic ring systems containing from 10-18 ring carbon atoms. Aryl groups include phenyl and polynuclear aromatics e.g., naphthyl, anthracenyl, phenanthrenyl, azulenyl and the like. The aryl group also includes groups such as ferrocenyl. Aryl groups may be unsubstituted or mono or polysubstituted with electron withdrawing or/and electron donating groups. A preferred aryl group is phenyl, which may unsubstituted or mono or polysubstituted with electron withdrawing or/and electron donating groups.

The term “aryl alkyl” as used herein alone or in combination with other term (s) means an alkyl group as defined herein carrying an aryl substitutent as defined herein. Preferred aryl alkyl groups are aryl-C₁-C₆-alkyl, aryl-C₁-C₃-alkyl, C₆-C₁₀-aryl-alkyl, C₈-C₁₀-aryl-C₁-C₈-alkyl, C₆-C₁₀-aryl-C₁-C₃-alkyl. More preferred aryl alkyl groups are phenyl-C₁-C₈-alkyl and phenyl-C₁-C₃-alkyl. Even more preferred aryl alkyl groups include, for example, benzyl, phenylethyl, phenylpropyl, phenylisopropyl, phenylbutyl, diphenylmethyl, 1,1-diphenylethyl, 1,2-diphenylethyl, and the like. Most preferred is benzyl.

The term “alkenyl” (alone or in combination with another term(s)) means a straight- or branched-chain alkenyl substituent containing at least one double bond and preferably containing from 2 to about 20 carbon atoms (C₂-C₂₀-alkenyl), more preferably from 2 to about 8 carbon atoms (C₂-C₈-alkenyl), and even more preferably from 2 to about 6 carbon atoms (C₂-C₆-alkenyl), most preferably 2 or 3 carbon atoms (C₂-C₃-alkenyl). The alkenyl group may be in the Z or E form. Alkenyl groups include vinyl, propenyl, 1-butenyl, isobutenyl, 2-butenyl, 1-pentenyl, (Z)-2-pentenyl, (E)-2-pentenyl, (Z)-4-methyl-2-pentenyl, (E)-4-methyl-2-pentenyl, pentadienyl, e.g., 1, 3 or 2,4-pentadienyl, and the like.

The term “alkynyl” (alone or in combination with another term(s)) means a straight- or branched-chain alkynyl substituent containing at least one triple bond and preferably containing from 2 to about 20 carbon atoms (C₂-C₂₀-alkynyl), more preferably from 2 to about 8 carbon atoms (C₂-C₈-alkynyl), and even more preferably from 2 to about 6 carbon atoms (C₂-C₆-alkynyl), most preferably 2 or 3 carbon atoms (C₂-C₃-alkynyl). The alkynyl group includes ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like.

The term “cycloalkyl” when used alone or in combination with another term (s) means a cycloalkyl group containing from 3 to 18 ring carbon atoms (C₃-C₁₈-cycloalkyl), preferably from 6 up to 10 ring carbon atoms (C₃-C₁₀-cycloalkyl), more preferably from 3 up to 6 ring carbon atoms. The cycloalkyl groups may be monocyclic, bicyclic, tricyclic, or polycyclic, and the rings may be fused. The cycloalkyl may be completely saturated or partially saturated. Examples of cycloalkyl groups include cyclopropyl, cyclobutyl, cyclopentyl, cyclohexyl, cycloheptyl, cyclooctyl, cyclodecyl, cyclohexenyl, cyclopentenyl, cyclooctenyl, cycloheptenyl, decalinyl, hydroindanyl, indanyl, fenchyl, pinenyl, adamantyl, and the like. The cycloalkyl group includes the cis or trans forms. Cycloalkyl groups may be unsubstituted or mono or polysubstituted with electron withdrawing or/and electron donating groups. In a bridged bicyclic cycloalkyl group, the substituents may either be in endo or exo positions.

The term “cycloalkyl alkyl” as used herein alone or in combination with other term(s) means an alkyl group as defined herein carrying a cycloalkyl substitutent as defined herein. Preferred cycloalkyl alkyl groups are cycloalkyl-C₁-C₆-alkyl, cycloalkyl-C₁-C₃-alkyl, C₆-C₁₀-cycloalkyl-alkyl, C₆-C₁₀-cycloalkyl-C₁-C₆-alkyl, C₆-C₁₀-cycloalkyl-C₁-C₃-alkyl. A more preferred cycloalkyl alkyl group is selected from cyclohexyl-C₁-C₆-alkyl and cyclohexyl-C₁-C₃-alkyl.

The term “halo” or “halogen” includes fluoro, chloro, bromo, and iodo.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Examples of haloalkyls include chloromethyl, 1-bromoethyl, fluoromethyl, difluoromethyl, trifluoromethyl, 1,1,1-trifluoroethyl, and the like. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), 1,1,1-trifluoroethoxy, and the like. It should be recognized that if a substituent is substituted by more than one halogen radical, those halogen radicals may be identical or different (unless otherwise stated).

The terms “electron-withdrawing” and “electron donating” refer to the ability of a substituent to withdraw or donate electrons, respectively, relative to that of hydrogen if the hydrogen atom occupied the same position in the molecule. These terms are well understood by one skilled in the art and are discussed in Advanced Organic Chemistry, by J. March, John Wiley and Sons, New York, N.Y., pp. 16-18 (1985) and the discussion therein is incorporated herein by reference. Electron withdrawing groups include halo, including bromo, fluoro, chloro, iodo; nitro, carboxy, alkenyl, alkynyl, formyl, carboxyamido, aryl, quaternary ammonium, haloalkyl such as trifluoromethyl, aryl alkanoyl, carbalkoxy and the like. Electron donating groups include such groups as hydroxy, alkoxy, including methoxy, ethoxy and the like; alkyl, such as methyl, ethyl, and the like; amino, alkylamino, dialkyl amino, aryloxy such as phenoxy, mercapto, alkylthio, alkylmercapto, disulfide (alkyldithio) and the like. One of ordinary skill in the art will appreciate that some of the aforesaid substituents may be considered to be electron donating or electron withdrawing under different chemical conditions. Moreover, the present invention contemplates any combination of substituents selected from the above-identified groups.

The electron donating or/and electron withdrawing groups may independently be present in any one of the substituents in Formula (I), (II) or/and (III) e.g., in R, R₁, R₂, R₃, R₄, R₅, R₆, R′6, R₇, R₈, R₉ or/and R₁₀ as defined herein.

The at least one electron withdrawing or/and at least one electron donating group is preferably selected independently from halo, alkyl, alkenyl, alkynyl, nitro, carboxy, formyl, carboxyamido, aryl, quaternary ammonium, haloalkyl, aryl alkanoyl, hydroxy, alkoxy, carbalkoxy, amino, alkylamino, dialkylamino, aryloxy, mercapto, alkylthio, alkylmercapto, disulfide, alkanoyl, amino alkyl, aryloyl, cyano, sulfonyl, sulfoxide, heterocyclic, guanidine, sulfonium salts, mercaptoalkyl, and alkyldithio.

The term “sulfide” encompasses mercapto, mercapto alkyl and alkylthio, while the term disulfide encompasses alkyldithio.

In the compounds of the present invention, the at least one electron withdrawing or/and at least one electron donating group is more preferably selected independently from halo, alkyl, alkenyl, alkynyl, nitro, carboxy, formyl, carboxyamido, aryl, quaternary ammonium, haloalkyl, aryl alkanoyl, hydroxy, alkoxy, carbalkoxy, amino, alkylamino, dialkylamino, aryloxy, mercapto, alkylthio, alkylmercapto, and disulfide.

Even more preferably, the at least one electron withdrawing or/and at least one electron donating group is selected from halo, C₁-C₆-alkyl, C₂-C₆-alkenyl, C₁-C₆-alkynyl, nitro, carboxy, formyl, carboxyamido, C₆-C₁₀-aryl, quaternary ammonium, C₁-C₆-haloalkyl, C₆-C₁₀-aryl C₂-C₆-alkanoyl, hydroxy, C₁-C₆-alkoxy, C₂-C₆-carbalkoxy, amino, C₁-C₆-alkylamino, C₁-C₆-dialkylamino, C₆-C₁₀-aryloxy, mercapto, C₁-C₆-alkylthio, C₁-C₆-alkylmercapto, and disulfide.

Even more preferably, the electron withdrawing or/and electron donating groups may also be independently selected from halo, C₁-C₆-alkoxy, nitro, carboxy, formyl, carboxyamido, quaternary ammonium, hydroxy, amino, mercapto, and disulfide.

Most preferred electron withdrawing or/and electron donating groups are independently selected from halo such as fluoro and C₁-C₆-alkoxy such as methoxy and ethoxy.

The term “carbalkoxy” as used herein alone or in combination with other term(s) means an —CO—O-alkyl, wherein alkyl is as defined herein, taking into account that the —CO—O— group provides one carbon atom in addition to those of the alkyl group. The carbalkoxy group preferably contains from 2 to about

20 carbon atoms (C₂-C₂₀-carbalkoxy), more preferably from 2 to about 8 carbon atoms (C₂-C₈-carbalkoxy), even more preferably from 2 to about 6 carbon atoms (C₂-C₆-carbalkoxy), and most preferably from 2 to 3 carbon atoms (C₂-C₃-carbalkoxy).

The term “alkanoyl” as used herein alone or in combination with other term (s) means an alkanoyl group —CO-alkyl, wherein alkyl is as defined herein, taking into account that the —CO— group provides one carbon atom in addition to those of the alkyl group. The alkanoyl preferably contains from 2 to about 20 carbon atoms (C₂-C₂₀-alkanoyl), more preferably from 2 to about 8 carbon atoms (C₂-C₈-alkanoyl), even more preferably from 2 to about 6 carbon atoms (C₂-C₆-alkanoyl), and most preferably from 2 to 3 carbon atoms (C₂-C₃-alkanoyl). The alkanoyl group may be straight chained or branched. The alkanoyl groups include, for example, formyl, acetyl, propionyl, butyryl, isobutyryl, tertiary butyryl, pentanoyl and hexanoyl.

As employed herein, a heterocyclic group contains at least one heteroatom in the cyclic structure, preferably one, two, three or four heteroatoms. The at least one heteroatom may be independently selected from sulfur, nitrogen and oxygen. The heterocyclic groups contemplated by the present invention include heteroaromatics and saturated and partially saturated heterocyclic groups. The heterocyclics may be monocyclic, bicyclic, tricyclic or polycyclic and may be fused rings. The heterocyclics also include the so-called benzoheterocyclics. Heterocyclic groups may be unsubstituted or mono or polysubstituted with electron withdrawing or/and electron donating groups. The heterocyclic groups preferably contain up to 18 ring atoms and up to a total of 17 ring carbon atoms and may be unsubstituted or mono or polysubstituted with electron withdrawing or/and electron donating groups.

More preferably, the heterocyclic group may be independently selected from 5 or 6-membered monocyclic heterocyclic groups and may be unsubstituted or mono or polysubstituted with electron withdrawing or/and electron donating groups. The heterocyclic group may also be more preferably selected independently from furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indazolyl, purinyl, indolinyl, pyrazolindinyl, imidazolinyl, imadazolindinyl, pyrrolidinyl, furazanyl, N-methylindolyl, methylfuryl, pyridazinyl, pyrimidinyl, pyrazinyl, pyridyl, epoxy, aziridino, oxetanyl, azetidinyl, the N-oxides of the nitrogen containing heterocycles, such as the N-oxides of pyridyl, pyrazinyl, and pyrimidinyl and the like. Even more preferably, the heterocyclic moieties are those aforementioned heterocyclics which are monocyclic.

The heterocyclics may also be more preferably selected independently from thienyl, furyl, pyrrolyl, benzofuryl, benzothienyl, indolyl, oxazolyl, methylpyrrolyl, morpholinyl, pyridiyl, pyrazinyl, imidazolyl, pyrimidinyl, and pyridazinyl. Especially preferred heterocyclic are independently selected from furyl, oxazolyl, pyridyl, pyrazinyl, imidazolyl, pyrimidinyl, and pyridazinyl. The most preferred heterocyclics are independently selected from furyl, pyridyl and oxazolyl.

The monocyclic 5- or 6-membered heterocyclic groups in the compounds of the present invention are preferably of the Formula (IV):

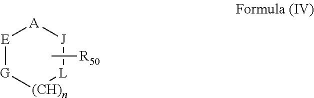

or those corresponding to a partially or fully saturated form thereof, wherein n is 0 or 1; and
R₅₀ is H, an electron withdrawing group or an electron donating group;
A, E, L, J and G are independently CH, or a heteroatom selected from the group consisting of N, O, S; but when n is 0, G is CH, or a heteroatom selected from the group consisting of NH, O and S with the proviso that at most two of A, E, L, J and G are heteroatoms.

When n is 0, the above heteroaromatic moiety is a five membered ring, while if n is 1, the heterocyclic moiety is a six membered monocyclic heterocyclic moiety.

If the ring depicted in Formula (IV) contains a nitrogen ring atom, then the N-oxide forms are also contemplated to be within the scope of the invention.

When R₂ or R₃ is a heterocyclic of Formula (IV), it may be bonded to the main chain by a ring carbon atom. When n is 0, R₂ or R₃ may additionally be bonded to the main chain by a nitrogen ring atom.

The term “heterocyclic alkyl” as used herein alone or in combination with other term(s) means an alkyl group as defined above carrying a heterocyclic substituent as defined above. Preferred heterocyclic alkyl groups are heterocyclic-C₁-C₆-alkyl, heterocyclic-C₁-C₃-alkyl, wherein the heterocyclic may be a preferred, more preferred or most preferred heterocyclic group as defined herein.

The term “alkyl heterocyclic” as used herein alone or in combination with other term(s) means a heterocyclic group as defined above carrying at least one alkyl substituent as defined above. Preferred alkyl heterocyclic groups are C₁-C₆-alkyl-heterocyclic, C₁-C₃-alkyl-heterocyclic, wherein the heterocyclic group may be a preferred, more preferred or most preferred heterocyclic group as defined herein.

The preferred compounds are those wherein n is 1, but di (n=2), tri (n=3) and tetrapeptides (n=4) are also contemplated to be within the scope of the invention.

In the ZY groups representative of R₂ or/and R₃, in the formula (I) or/and (II), Z may be O, S, S(O)ₐ, wherein a is 1-3, NR₄, NR′₆, PR₄ or a chemical bond; and Y may be hydrogen, alkyl, aryl, aryl alkyl, alkenyl, alkynyl, halo, heterocyclic, heterocyclic alkyl, alkyl heterocyclic, and Y may be unsubstituted or substituted with at least one electron donating group or/and at least one electron withdrawing group, provided that when Y is halo, Z is a chemical bond, or

ZY taken together may be NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR₄NR₅R₇ or N+R₅R₆R₇,

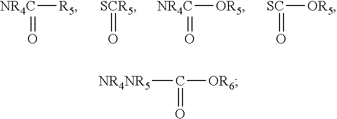

wherein R₄, R₅, R′₆, R₆, R₇, are as defined herein.

The ZY groups representative of R₂ or/and R₃ in the Formula (I) or/and (II) may be hydroxy, alkoxy, such as methoxy, ethoxy, aryloxy, such as phenoxy; thioalkoxy, such as thiomethoxy, thioethoxy; thioaryloxy such as thiophenoxy; amino; alkylamino, such as methylamino, ethylamino; arylamino, such as anilino; dialkylamino, such as, dimethylamino; trialkyl ammonium salt, hydrazino; alkylhydrazino and arylhydrazino, such as N-methylhydrazino, N-phenylhydrazino, carbalkoxy hydrazi no, aralkoxycarbonyl hydrazino, aryloxycarbonyl hydrazino, hydroxylamino, such as N-hydroxylamino (—NH—OH), alkoxy amino [(NHOR₁₅) wherein R₁₈ is alkyl], N-alkylhydroxylamino [(NR₁₈)OH wherein R₁₈ is alkyl], N-alkyl-β-alkylhydroxyamino, i.e., [N(R₁₅)OR₁₃ wherein R₁₈ and R₁₉ are independently alkyl], and O-hydroxylamino (—O—NH₂); alkylamido such as acetamido; trifluoroacetamido; alkoxyamino, (e.g., NH(OCH₃); and heterocyclicamino, such as pyrazoylamino.

In a preferred ZY group, Z is O, NR₄ or PR₄; Y is hydrogen or alkyl.

In another preferred embodiment,

### ZY is NR₄R₅R₇, NR₄OR₅, ONR₄R₇,

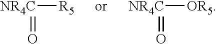

It is more preferred that ZY is NR₄OR₅, or ONR₄R₇.

Another more preferred ZY is N-hydroxyamino, N-alkylhydroxyamino, N-alkyl-O-alkyl hydroxyamino, O-alkylhydroxyamino, N-alkoxy-N-alkylamino, N-alkoxyamino, or N-carbalkoxy.

In Formula (I), R is preferably aryl or aryl alkyl, more preferably R is aryl alkyl, wherein R is unsubstituted or substituted with at least one electron donating group or/and at least one electron withdrawing group. R may be phenyl or benzyl, most preferably benzyl, wherein R is unsubstituted or substituted with at least one electron donating group or/and at least one electron withdrawing group. If R is substituted, R is preferably substituted on the aryl ring. In this embodiment, the at least one electron donating group or/and at least one electron withdrawing group is preferably halo, more preferably fluoro.

In Formulae (I), (II) or/and (III), R₁ is H or alkyl. More preferably, R₁ is alkyl, preferably containing from 1 to 6 carbon atoms, more preferably containing from 1 to 3 carbon atoms. Most preferably the R, group is methyl. R₁ may be unsubstituted or substituted with at least one electron donating group or/and at least one electron withdrawing group.

Further, it is preferred that one of R₂ and R₃ is hydrogen. It is more preferred that R₂ is hydrogen. Other preferred moieties of R₂ in Formula (I) are aryl such as phenyl, aryl alkyl such as benzyl, and alkyl. It is to be understood that the preferred groups of R₂ may be unsubstituted or mono or poly substituted with electron donating or/and electron withdrawing groups. It is preferred that the at least one electron withdrawing or/and at least one donating group in R₂ is independently alkoxy, N-hydroxyamino, N-alkylhydroxyamino, N-alkyl-O-alkyl hydroxyamino or O-alkylhydroxyamino, and especially methoxy or ethoxy.

In Formulae (I), (II) or/and (III), R₃ may be hydrogen, an alkyl group unsubstituted or substituted by at least an electron donating or/and at least one electron withdrawing group, an aryl group unsubstituted or substituted by at least an electron donating or/and at least one electron withdrawing group heterocyclic, heterocyclic alkyl, or ZY.

It is preferred that R₃ is hydrogen, alkyl unsubstituted or substituted by at least an electron donating or/and at least one electron withdrawing group, aryl which is unsubstituted or substituted by at least one electron donating group or/and at least one electron withdrawing group, heterocyclic, heterocyclic alkyl or ZY, wherein Z is O, NR₄ or PR₄; Y is hydrogen or alkyl; ZY is NR₄NR₅R₇, NR₄OR₅, ONR₄R₇,

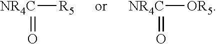

It is also preferred that R₃ is alkyl unsubstituted or substituted by at least an electron donating or/and at least one electron withdrawing group; or Z—Y, wherein Z—Y is as defined herein.

It is also preferred that R₃ is alkyl unsubstituted or substituted by at least an electron donating or/and at least one electron withdrawing group; NR₄OR₅, or ONR₄R₇, wherein R₄, R₅ and R₇ are as defined herein.

It is also preferred that R₃ is CH₂-Q, wherein Q is alkoxy especially containing 1-3 carbon atoms; or R₃ is NR₄OR₅ or ONR₄R₇, wherein R₄, R₅, and R₇ are as defined herein.

R₃ is also preferably alkyl which is unsubstituted or substituted with at least one alkoxy especially containing 1-3 carbon atoms.

R₃ is also preferably CH₂-Q, wherein Q is alkoxy preferably containing 1-3 carbon atoms, more preferably Q is ethoxy or methoxy.

R₃ is also preferably NR₄OR₅, or ONR₄R₇, wherein R₄, R₅ and R₇ are as defined herein, and R₄, R₅ and R₇ are as defined herein, e.g. N-alkoxy, N-alkoxy-N-alkylamino or N-carbalkoxy.

R₃ is also preferably heterocyclic, heterocyclic alkyl, or aryl, which may be unsubstituted or substituted with at least an electron donating or/and at least one electron withdrawing group. A most preferred heterocyclic in R₃ is furyl or oxazolyl.

R₃ is also preferably selected from the group consisting of hydrogen, alkyl, arylalkyl such as benzyl, alkoxy, alkoxyalkyl, aryl such as phenyl, heterocyclic, heterocyclic alkyl, N-alkoxy-N-alkylamino, N-alkoxyamino and N-carbalkoxy.

It is to be understood that the preferred groups of R₃ may be unsubstituted or mono or poly substituted with electron donating or/and electron withdrawing groups. It is preferred that the at least one electron withdrawing or/and at least one electron donating group in R₃ is independently alkoxy, N-hydroxyamino, N-alkylhydroxyamino, N-alkyl-O-alkyl hydroxyamino or O-alkylhydroxyamino, and especially methoxy or ethoxy.

R₄, R₅, R₆, R′ₛ, R₇ and R₈ are preferably independently hydrogen or alkyl.

R₄, R₅, and R₇ are preferably independently hydrogen or alkyl preferably containing 1-3 carbon atoms.

The most preferred aryl is phenyl. The most preferred halo is fluoro.

In the compounds of Formula (I), R is preferably aryl alkyl, wherein R is unsubstituted or substituted with at least one electron donating group or/and at least one electron withdrawing group.

In the compounds of Formula (I), R₁ is preferably alkyl which is unsubstituted or substituted with at least one electron donating group or/and at least one electron withdrawing group.

In the compounds of Formula (I), R₂ and R₃ is preferably independently hydrogen, alkyl which is unsubstituted or substituted by at least one electron donating group or/and at least one electron withdrawing group, aryl which is unsubstituted or substituted by at least one electron donating group or/and at least one electron withdrawing group, heterocyclic, heterocyclic aryl, or ZY; wherein Z is O, NR₄ or PR₁; and Y is hydrogen or

alkyl; or ZY is NR₄NR₅R₇, NR₄OR₅, ONR₄R₇,

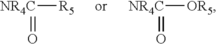

wherein R₄, R₅ and R₇ are as defined herein.

In the compounds of Formula (I), the preferred groups of R₂ and R₃ may be unsubstituted or mono or poly substituted with electron donating or/and electron withdrawing groups, such as alkoxy (e.g., methoxy, ethoxy, and the like), N-hydroxyamino, N-alkylhydroxyamino, N-alkyl-O-alkyl hydroxyamino and O-alkylhydroxyamino.

In the compounds of Formula (I), the at least one electron donating group or/and at least one electron withdrawing group in R₂ or/and R₃ is preferably independently hydroxy or alkoxy.

It is more preferred that in the compounds of Formula (I), R₂ is hydrogen.

In the compounds of Formula (II), R₁ is preferably methyl.

In preferred compounds of Formula (II), R₃ is hydrogen or alkyl unsubstituted or substituted by at least one electron donating group or/and at least one electron withdrawing group; or R₃ is heterocyclic, heterocyclic alkyl, or Z—Y, wherein Z—Y and heterocyclic are as defined herein.

In other preferred compounds of Formula (II), R₃ is an alkyl group which is unsubstituted or substituted by at least one electron donating group or/and at least one electron withdrawing group, NR₄OR₅ or ONR₄R₇, wherein R₄, R₅ and R₇ are as defined herein and wherein the at least one electron donating group or/and at least one electron withdrawing group is preferably selected from hydroxy and alkoxy.

In further preferred compounds of Formula (II), R₃ is CH₂-Q, wherein Q is alkoxy preferably containing 1-3 carbon atoms, more preferably methoxy, or R₃ is NR₄OR₅ or ONR₄R₇ wherein R₄, R₅ and R₇ are independently hydrogen or alkyl containing 1-3 carbon atoms.

In other preferred compounds of Formula (II), R₃ is —CH₂-Q, wherein Q is alkoxy containing 1 to 3 carbon atoms.

In the compounds of Formula (II), Ar is preferably phenyl unsubstituted or substituted with at least one halo, preferably with at least one fluoro. More preferably Ar in Formula (II) is unsubstituted phenyl.

In preferred compounds of Formula (III), R₉ is hydrogen or fluoro, R₃ is selected from the group consisting of methoxymethyl, phenyl, N-methoxy-N-methylamino, and N-methoxyamino, and R₁ is methyl.

The most preferred compounds of the present invention include:
(R)-2-acetamido-N-benzyl-3-methoxy-propionamide;
(R)-2-acetamido-N-benzyl-3-ethoxy-propionamide;
O-methyl-N-acetyl-D-serine-m-fluorobenzyl-amide;
O-methyl-N-acetyl-D-serine-p-fluorobenzyl-amide;
N-acetyl-D-phenylglycine benzylamide;
D-1,2-(N,O-dimethylhydroxylamino)-2-acetamide acetic acid benzylamide;
D-1,2-(O-methylhydroxylamino)-2-acetamido acetic acid benzylamide;
D-α-acetamido-N-(2-fluorobenzyl)-2-furanacetamide;
D-α-acetamido-N-(3-fluorobenzyl)-2-furanacetamide.

It is to be understood that the various combinations and permutations of the Markush groups of R₁, R₂, R₃, R and n described herein are contemplated to be within the scope of the present invention. Moreover, the present invention also encompasses compounds and compositions which contain one or more elements of each of the Markush groupings in R₁, R₂, R₃, n and R and the various combinations thereof. Thus, for example, the present invention contemplates that R₁ may be one or more of the substituents listed hereinabove in combination with any and all of the substituents of R₂, R₃, and R with respect to each value of n.

More preferred is a compound of Formula (I), (II) or/and (III) in the R configuration, preferably substantially enantiopure, wherein the substituent R is benzyl which is unsubstituted with at least one halo group, wherein R₃ is CH₂-Q, wherein Q is alkoxy containing 1-3 carbon atoms and wherein R₁ is methyl. Preferably R is unsubstituted benzyl or benzyl substituted with at least one halo group which is a fluoro group.

Depending upon the substituents, the present compounds may form addition salts as well. All of these forms are contemplated to be within the scope of this invention including mixtures of the stereoisomeric forms.

The manufacture of compounds utilized in the present invention is described in U.S. Pat. Nos. 5,378,729 and 5,773,475, and in the international application PCT/EP 2005/010603 the contents of which are incorporated by reference.

The compounds utilized in the present invention are useful as such as depicted in the Formulae (I), (II) or/and Op or can be employed in the form of salts in view of its basic nature by the presence of the free amino group.

Thus, the compounds of Formulae (I), (II) or/and (III) form salts with a wide variety of acids, inorganic and organic, including pharmaceutically acceptable acids. The salts with therapeutically acceptable acids are of course useful in the preparation of formulation where enhanced water solubility is most advantageous.

These pharmaceutically acceptable salts have also therapeutic efficacy. These salts include salts of inorganic acids such as hydrochloric, hydroiodic, hydrobromic, phosphoric, metaphosphoric, nitric acid and sulfuric acids as well as salts of organic acids, such as tartaric, acetic, citric, malic, benzoic, perchloric, glycolic, gluconic, succinic, aryl sulfonic, (e.g., p-toluene sulfonic acids, benzenesulfonic), phosphoric, malonic, and the like.

The SE treated by a method of the present embodiment is at least partially refractory or substantially refractory against at least one anti-epileptic drug, for example a benzodiazepine, barbiturate or anticonvulsive other than a compound of Formula (I), (II) or (III). In a particular embodiment, the at least one anti-epileptic drug to which the SE is refractory is selected from the group consisting of diazepam, lorazepam, midazolam, phenobarbital, carbamazepine, phenyloin, fosphenyloin, oxcarbazepine, lamotrigine, gabapentin, pregabalin, valproic acid, pentobarbital, thiopental, propofol and pharmaceutically acceptable salts thereof.

A compound of Formulas (I), (II) or (III), for example lacosamide, is used in a therapeutically effective amount.

The physician will determine the dosage of the present therapeutic agents which will be most suitable and it will vary with the form of administration and the particular compound chosen, and furthermore, it will vary with the patient under treatment, the age of the patient, the type of malady being treated. He will generally wish to initiate treatment with small dosages substantially less than the optimum dose of the compound and increase the dosage by small increments until the optimum effect under the circumstances is reached. When the composition is administered orally, larger quantities of the active agent will be required to produce the same effect as a smaller quantity given parenterally. The compounds are useful in the same manner as comparable therapeutic agents and the dosage level is of the same order of magnitude as is generally employed with these other therapeutic agents.

In one embodiment, the compounds of the present invention are administered in amounts ranging from about 1 mg to about 100 mg per kilogram of body weight per day, more preferably in amounts ranging from about 1 mg to about 10 mg per kilogram of body weight per day. This dosage regimen may be adjusted by the physician to provide the optimum therapeutic response. Patients in need thereof may be treated with doses of the compound of the present invention of at least 50 mg/day, preferably of at least 200 mg/day, more preferably of at least 300 mg/day, still more preferably of at least 400 mg/day and most preferably of at least 600 mg/day. Generally, a patient in need thereof may be treated with doses at a maximum of 6 g/day, more preferably a maximum of 1 g/day, still more preferably a maximum of 600 mg/day, and most preferably a maximum of 800 mg/day. In some cases, however, higher or lower doses may be needed.

In another preferred embodiment, the daily doses are increased until a predetermined daily dose is reached which is maintained during the further treatment.

Doses expressed herein on a daily basis, for example in mg/day, are not to be interpreted as requiring a once-a-day frequency of administration. For example, a dose of 300 mg/day can be given as 100 mg three times a day, or as 600 mg every second day.

More typically, in an emergency situation, a compound of Formulas (I), (II) or (III), for example lacosamide, is administered not on a daily basis but pro re nata (p.r.n.), typically after onset of SE. A typical single dose of lacosamide, for example, is an amount of about 50 to about 500 mg. Such administration can occur, for example, at any time from immediately after onset until about 60 minutes after onset or even later. In various embodiments administration occurs about 10, about 15, about 20, about 30, about 45 or about 60 minutes after onset.

Refractory SE, especially where the SE is of the generalized convulsive type, is an emergency situation and it is generally important to administer medication as soon as possible after onset. Thus in a particular embodiment a compound of Formulas (I), (II) or (III), for example lacosamide, is administered immediately after onset of SE or as soon as possible thereafter.

A compound of Formulas (I), (II) or (III), for example lacosamide, can be used in first line treatment of refractory SE, for example where prior SE episodes have proven refractory to other treatments.

Alternatively, a compound of Formulas (I), (II) or (III), for example lacosamide, can be used in second line treatment of refractory SE, wherein resistance has already become apparent following a preceding first line treatment, such as with one or more benzodiazepines, barbiturates or anticonvulsants other than compounds of Formula (I), in particular phenyloin, fosphenyloin or valproic acid.

Typically in second line treatment, a compound of Formulas (I), (II) or (III) is administered at least about 10 minutes, for example at least about 15, at least about 20, at least about 30, at least about 45 or at least about 60 minutes, after onset of SE. This administration can occur independently of the time when a seizure or seizure cluster becomes refractory to a first line treatment, but in one embodiment occurs immediately or as soon as possible after resistance becomes apparent to the first line treatment.

In yet another embodiment, several divided doses may be administered daily. For example, three doses per day may be administered, preferably two doses per day. It is more preferred to administer a single dose per day.

In yet another preferred embodiment, an amount of the compounds of the present invention may be administered which results in a plasma concentration of 0.1 to 15 μg/ml (trough) and 5 to 18.5 μg/ml (peak), calculated as an average over a plurality of treated subjects, intravenous administration in emergency treatment might result in peak plasmid levels of up to 30 μg/ml.

The compounds of Formulae (I), (II) or/and (III) may be administered in a convenient manner, such as by oral, intravenous (where water soluble), intramuscular, intrathecal, rectal (e.g. suppository, gel, liquid, etc.) or subcutaneous routes. Oral, rectal or/and intravenous (i.v.) administration is preferred. In emergency treatment, i.v. administration is most preferred.

The pharmaceutical composition of the present invention may be prepared for the treatment regimen as described above, in particular for the treatment with doses as described above, to effect plasma concentrations as described above, for administration periods or/and administration routes as specified in the embodiments of the present invention as described above.

The compounds of Formulae (I), (II) or/and (III) may be orally administered, for example, with an inert diluent or with an assimilable edible carrier, or it may be enclosed in hard or soft shell gelatin capsules, or it may be compressed into tablets, or it may be incorporated directly into the food of the diet. For oral therapeutic administration, the active compound of Formulae (I), (II) or/and (III) may be incorporated with excipients and used in the form of ingestible tablets, buccal tablets, troches, capsules, elixirs, suspensions, syrups, wafers, and the like. Such compositions and preparations should contain at least 1% of active compound of Formulae (I), (II) or/and (III). The percentage of the compositions and preparations may, of course, be varied and may conveniently be between about 5 to about 80% of the weight of the unit. The amount of active compound of Formulae (I), (II) or/and (III) in such therapeutically useful compositions is such that a suitable dosage will be obtained. Preferred compositions or preparations according to the present invention contains between about 10 mg and 6 g active compound of Formulae (I), (II) or/and (III), for example about 50 to about 1000 mg, or about 100 to about 800 mg, of the compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations. For example, sustained release dosage forms are contemplated wherein the active ingredient is bound to an ion exchange resin which, optionally, can be coated with a diffusion barrier coating to modify the release properties of the resin.

The active compound may also be administered parenterally or intraperitoneally. Dispersions can also be prepared in glycerol, liquid, polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of preparing sterile powders for the manufacture of sterile injectable solutions, the preferred methods of preparation are vacuum drying, or freeze-drying optionally together with any additional desired ingredient.

As used herein, “pharmaceutically acceptable carrier” includes any and all solvents, dispersion media, coatings, antibacterial and antifungal agent, isotonic and absorption delaying agents for pharmaceutical active substances as well known in the art. Except insofar as any conventional media or agent is incompatible with the active ingredient, its use in the therapeutic compositions is contemplated. Supplementary active ingredients can also be incorporated into the compositions.

It is especially advantageous to formulate parenteral compositions in dosage unit form or ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specifics for the novel dosage unit forms of the invention are dictated by and directly dependent on (a) the unique characteristics of the active material an the particular therapeutic effect to be achieved, and (b) the limitations inherent in the art of compounding such as active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore described. A unit dosage form can, for example, contain the principal active compound in amounts ranging from about 10 mg to about 6 g. Expressed in proportions, the active compound is generally present in from about 1 to about 750 mg/ml of carrier. In the case of compositions containing supplementary active ingredients, the dosages are determined by reference to the usual dose and manner of administration of the said ingredients.

As used herein the term “patient” or “subject” refers to a warm blooded animal, and preferably mammals, such as, for example, cats, dogs, horses, cows, pigs, mice, rats and primates, including humans. The preferred patient is a human.

The term “treat” refers to either relieving the pain associated with a disease or condition, to providing partial to complete relieve of the patient's disease or condition, or alleviating the patient's disease or condition. More specifically, unless the context demands otherwise, the term “treat,” “treating” or “treatment” herein includes preventive or prophylactic use of a medication in a subject at risk of, or having a prognosis including, a refractory epileptic condition, as well as use of such a compound in a subject already experiencing a refractory epileptic condition, as a therapy to alleviate, relieve, reduce intensity of or eliminate such a condition or an underlying cause thereof. In a particular aspect, administration of a medication according to a method of the invention is post-onset of SE. At the time of administration the SE may already be refractory or, based on prior episodes or on the duration of the seizures, may have a prognosis of becoming refractory.

The compounds of the present invention are administered to a patient suffering from the aforementioned type of disorder in an effective amount. These amounts are equivalent to the therapeutically effective amounts described hereinabove.

The invention is further illustrated by the following figure and example.

### FIGURE LEGENDS

FIG. 1: The effects of lacosamide in the self-sustained status epilepticus model for treatment-resistant status epilepticus.

FIG. 2: Effect of early treatment on number of SRS/week.

FIG. 3: Effect of late treatment on number of seizures/week.

### EXAMPLES

While standard anti-epileptic drugs can work relatively well if given very early in the course of SE, they typically lose their efficacy as seizures continue, especially if seizures continue for more than about 30 minutes.

These clinical features can be reproduced experimentally, using the perforant path stimulation model and the lithium/pilocarpine model of status epilepticus. Lacosamide was studied in these two models by administration at a defined time after onset of experimentally induced SE, at which time the standard drugs have a reduced efficacy or are even inactive. For example, Mazarati et al. (1999, Neurosci Lett. 265:187-190) stated that during the course of self-sustaining status epilepticus (SSSE) in the perforant paths stimulation (PPS) model, resistance to standard anticonvulsants developed progressively: diazepam and phenyloin were highly effective when given before or a onset of SSSE, but lost their effectiveness when administration was delayed.

Lacosamide was studied in the perforant path stimulation model and the lithium/pilocarpin model of status epilepticus. Lacosamide was studied in these two models for treatment of refractory status epilepticus, wherein lacosamide is administered at a defined period after onset of the experimentally induced status epilepticus, at which time the standard drugs have a reduced efficacy or are even inactive.

The following examples illustrate anticonvulsive efficacy of lacosamide, alone and in combination with diazepam, in models for refractory SE.

### Example 1

### Perforant Path Stimulation Model

Male Wistar rats were implanted with a stimulating electrode into the angular bundle of the perforant path and a recording electrode into the granule cell layer of the dentate gyrus. Perforant path stimulation (PPS) was delivered for 30 or 60 minutes with the following parameters: 10 s, 20 Hz trains of 1 ms, 30 V pulses delivered every minute together with continuous 2 Hz stimulation with the same parameters.

Lacosamide was injected intraperitoneally 40 minutes after the end of PPS at a dose of 50 mg/kg. The following indices were used to quantify seizure activity: cumulative seizure time (duration of SSSE, subtracting interictal time) and the number of seizure episodes. In addition the number of spontaneous seizures was measured 6 months following induction of SSSE in order to assess status epilepticus induced epileptogenesis.

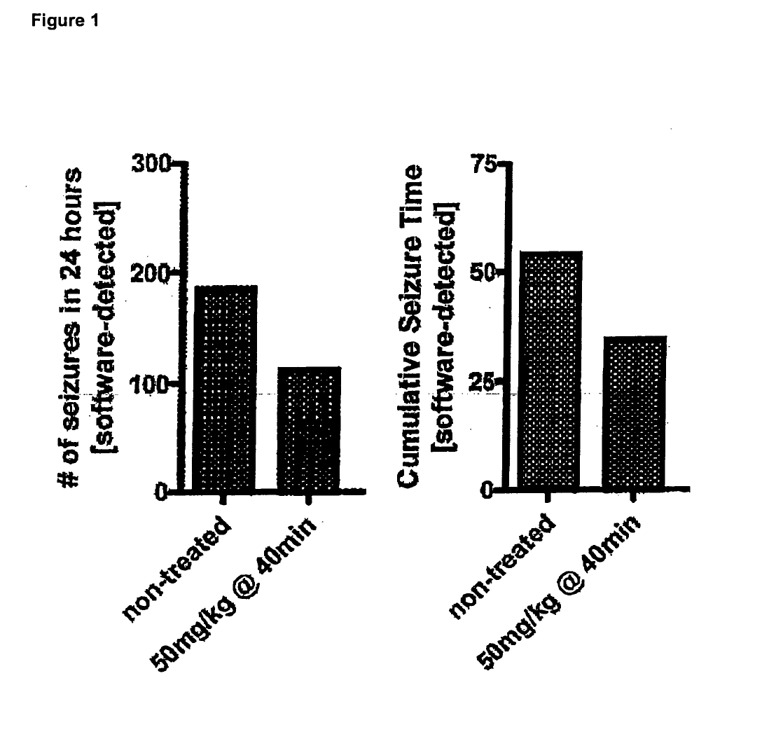

When lacosamide treatment was initiated 40 minutes after PPS, a substantial reduction in both seizure frequency and cumulative seizure duration was obtained, as shown in FIG. 1.

### Example 2

### Lithium/Pilocarpine Model

Rats received 3 mmol/kg lithium 20-24 hours prior to administration of 40 mg/kg pilocarpine. Lacosamide treatment was initiated after 10 minutes of high amplitude rapid continuous spiking on EEG. This is a time that has previously been demonstrated to be refractory to treatment with standard clinical anti-SE drugs in this model (see, for example, a study of response to diazepam by Walton & Treiman (1988) Exp. Neurol. 101:267-275).

Treatment with lacosamide (50 mg/kg) reduced motor seizure symptoms under conditions where standard anti-status drugs were completely inactive.

Another group of rats received 50 mg/kg lacosamide followed 5 minutes later by 20 mg/kg diazepam. Full control of seizures was achieved in all rats by this combination treatment.

It is concluded that the compounds of the present invention, in particular lacosamide, or a combination of the compounds of the present invention, in particular of lacosamide, with one or more further drug used in the treatment of SE, such as benzodiazepines, anticonvulsants or barbiturates, preferably a benzodiazepine, in particular diazepam, is suitable for the treatment of refractory status epilepticus or for the treatment of a long-lasting SE which is or becomes refractory in the course of its duration.

### Example 3

### Long Term Effects of Lacosamide (Disease-Modifying Effects)

SSSE was induced in rats as described in Example 1. After SSSE induction, and at least 6 months wait (“silent period”) the animals were placed in EEG/telemetry/videotape continuously for two weeks for chronic EEG and video monitoring, but the second week, which was more remote from anaesthesia and surgery, was used to calculate seizure frequency (24 hours/day×7). Electrographic seizures were captured by the Harmony software, and were confirmed by offline manual review of the EEG and videotapes. The following indices were counted: total number of spikes of seizures for 7 days of observation, mean seizure duration, light/dark distribution.

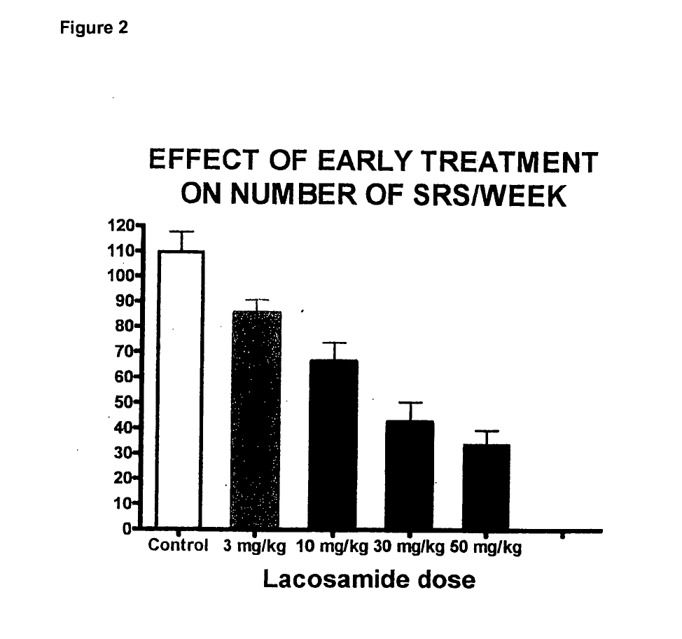

Treatment of status epilepticus 10 min after perforant path stimulation with lacosamide had significant effects on several of the long-term consequences of status epilepticus. The number of spontaneous recurrent seizures (SRSs) per week (FIG. 2) was reduced from 110±8 in vehicle-treated animals to 85±5 in rats receiving 3 mg/kg of lacosamide, and in animals treated with 10 mg/kg, 30 mg/kg or 50 mg/kg respectively, it was 66±8, 42±8 and 34±6.

This disease-modifying effect of small doses of lacosamide was also observed when looking at spike frequency, which was reduced from 9534±1114 spikes/week in controls to 7557±1945 spikes/week in the 3 mg/kg group, and to 3536±380, 2969±542, and 2588±370 spikes/week in the mg/kg, 30 mg/kg and 50 mg/kg groups, respectively.

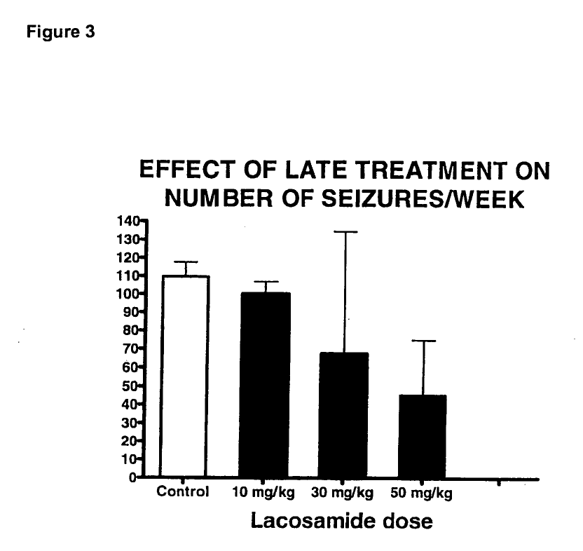

Treatment 40 min after perforant path stimulation reduced the number of animals showing spontaneous recurrent seizures from 6/6 to 3/9 in the two higher dosage treatment groups combined (p<0.05). When the two highest treatment groups were combined, they reduced seizure numbers from 110±8 to 55±32 seizures per week. When individual treatments were analysed, the number of seizures per week went from 110±8 to 100±7 (lacosamide 10 mg/kg), 67±67 (lacosamide 30 mg/kg) and 45±29 (lacosamide 50 mg/kg), but these changes were not statistically significant (FIG. 3).

However, the median number of seizures in the 30 mg/kg and 50 mg/kg groups was 0, reflecting the fact that the majority of animals had no SRSs.

Lacosamide was effective as an anticonvulsant when given 10 min after perforant path stimulation in the development of status epilepticus, and at doses 10 mg/kg and above, it reduced the number of seizures, as well as the cumulative time spent seizing after treatment.

Chronically, early lacosamide treatment (10 min after perforant path stimulation) reduced the frequency of spontaneous recurrent seizures and reduce spike frequency.

Treatment of established, self-sustaining status epilepticus 40 min after perforant path stimulation (late treatment) produced a non-significant reduction in the number of seizures.

Treatment with lacosamide at high dose (30-50 mg/kg) reduced the incidence of chronic SRSs, and the frequency of those SRSs, suggesting a disease-modifying effect on chronic epileptogenesis.

Early treatment reduced the severity of the subsequent chronic epilepsy, a disease-modifying effect. After late treatment, a disease-modifying effect was observed when the two high-dose groups were combined for analysis.

1-72. (canceled)

73. A method for alleviating and/or treating epileptogenesis comprising administering lacosamide to a subject in need thereof.

74. The method of claim 73, wherein the epileptogenesis is related to status epilepticus.

75. The method of claim 74, wherein the epileptogenesis is related to refractory status epilepticus.

76. The method of claim 74, wherein lacosamide is administered after onset of status epilepticus.

77. The method of claim 76, wherein lacosamide is administered at least about 10 minutes after onset of status epilepticus.

78. The method of claim 76, wherein lacosamide is administered at least about 30 minutes after onset of status epilepticus.

79. The method of claim 73, further comprising administering at least one benzodiazepine to the subject.

80. The method of claim 79, wherein the at least one benzodiazepine is diazepam, lorazepam and/or midazolam.

81. The method of claim 73, wherein the epileptogenesis comprises development of an epileptic condition comprising focal seizures and/or generalized seizures.

82. The method of claim 79, wherein lacosamide and the at least one benzodiazepine are administered to the subject in a single dosage or separate dosages forms.

83. The method of claim 82, wherein the separate dosages forms are administered simultaneously or sequentially.

84. The method of claim 82, wherein the separate dosages forms are administered by the same route or by different routes.

85. The method of claim 82, wherein the separate dosages forms are co-packaged or co-presented in separate packaging but co-marketed or co-promoted for use together.

86. The method of claim 73, wherein lacosamide is administered in a dosage amount of 50 mg/day to 1000 mg/day.

87. The method of claim 73, wherein lacosamide is administered in a dosage amount of 200 mg/day to 600 mg/day.

88. The method of claim 73, wherein lacosamide is administered in a dosage amount of at least about 50 mg/day.

89. The method of claim 73, wherein lacosamide is administered in a dosage amount of at least about 100 mg/day.

90. The method of claim 73, wherein lacosamide is administered in a dosage amount of at least about 200 mg/day.

91. The method of claim 73, wherein lacosamide is administered in a dosage amount of up to about 600 mg/day.
